(19) United States
(12) Patent Application Publication
(10) Pub. No.: US 2005/0255098 A1
(43) Pub. Date: Nov. 17, 2005

(54) Methods of treating traumatic spinal cord injury

(76) Inventors: Steven D. Rosen, San Francisco, CA (US); Linda J. Noble, San Francisco, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/844,035

(22) Filed: May 11, 2004

(51) Int. Cl. A61K 39/395

(52) U.S. Cl. 424/130.1

(57) ABSTRACT

The present invention provides methods of treating traumatic spinal cord injury, methods of reducing cell-mediated demyelination of long descending fiber tracts or local circuits in the spinal cord following traumatic spinal cord injury, and methods for improving or restoring locomotor recovery and/or fine motor movement in an individual following spinal cord injury. The present invention further provides compositions for use in the methods. The methods generally involve administering to an individual in need thereof an effective amount of an L-selectin antagonist.

### FIELD OF THE INVENTION

The present invention is in the field of traumatic spinal cord injury, and in particular to the use of L-selectin antagonists to treat traumatic spinal cord injury.

### BACKGROUND OF THE INVENTION

Spinal cord injuries occur in approximately 12,000 to 15,000 people per year in the United States alone. About 10,000 of these people are permanently paralyzed, and many of the rest die as a result of their injuries. Spinal cord injuries often result in reduced locomotor movement, fine motor movement, sensory functions, urinary elimination, and so forth. Traumatic spinal cord injuries are typically caused by traffic accidents, athletic accidents, falls and drops from heights, assaults, and the like.

Methylprednisolone has historically served as the “standard of care” for the treatment of the acutely spinal cord injured patient. The mechanism by which methylprednisolone confers neuroprotection is not clear and has been attributed to its ability to decrease swelling, inflammation, free radical generation, and glutamate release. However, there is growing controversy regarding the effectiveness of methylprednisolone. The American Association of Neurological Surgeons/Congress of Neurological Surgeons Joint Section of Disorders of the Spine and Peripheral Nerves has recently recommended that the use of methylprednisolone be considered optional. Importantly, high dose methylprednisolone treatment is associated with complications including an increased frequency of gastric bleeding and wound infection.

There is a need in the art for methods for treating traumatic spinal cord injury. The present invention addresses this need.

Literature

U.S. Pat. No. 5,227,369; U.S. Pat. No. 6,432,404; Faden et al. (1984) J. Neurosurg. 60:712-717; Guney et al. (1998) Neurosurg. Rev. 21:265-269; Paxton et al. (1995) J. Trauma 38:920-923; Blight (1985) Central Nervous System Trauma 2:299-315; Blight (1992) J. Neurotrauma 9 Suppl 1:S83-91; Blight et al. (1995) Brain 118 (Pt 3):735-752; Blight et al. (1997) J. Neurotrauma 14:89-98; Bethea et al (1998) J. Neuroscience 18:3251-3260; Dusart and Schwab (1994) Eur. J Neurosci. 6:712-724; Streit et al. (1998) Experimental Neurology 152:74-87; Dijkstra et al. (1994) J. Immunol. Methods 174:21-23; Hirschberg et al. (1994) J. Immunol. Methods 174:21-23; Blight (1994) Neuroscience 60:263-273; Giulian and Robertson (1990) Annals of Neurology 27:33-42; Gunnarsson and Fehlings (2003) Curr Opin Neurol 16:717-723; McDonald and Sadowsky (2002) Lancet 359:417-425.

### SUMMARY OF THE INVENTION

The present invention provides methods of treating traumatic spinal cord injury, methods of reducing cell-mediated demyelination of long descending fiber tracts or local circuits in the spinal cord following traumatic spinal cord injury, and methods for improving or restoring locomotor recovery and/or fine motor movement in an individual following spinal cord injury. The present invention further provides compositions for use in the methods. The methods generally involve administering to an individual in need thereof an effective amount of an L-selectin antagonist.

### BRIEF DESCRIPTION OF THE DRAWINGS

FIG. 1 is a graph depicting locomotor recovery in L-selectin knockout animals and wild-type animals, following traumatic spinal cord injury.

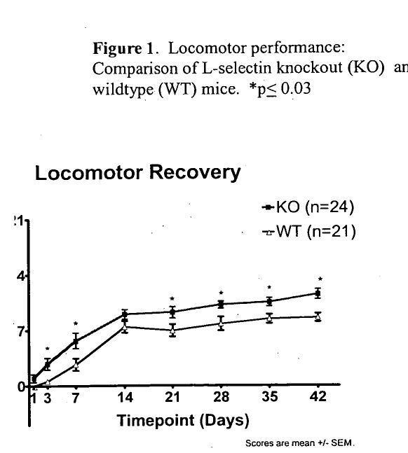

FIG. 2 is a graph depicting the percent of residual white matter at the lesion epicenter in L-selectin knockout animals and wild-type animals, following traumatic spinal cord injury.

FIG. 3 provides an amino acid sequence of human L-selectin (SEQ ID NO:1).

### DEFINITIONS

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

The terms “individual,” “host,” “subject,” and “patient,” used interchangeably herein, refer to a mammal, including primates, rodents, livestock, mammalian pets, horses, etc. In some embodiments, an individual is a human.

The term “binds specifically,” in the context of antibody binding, refers to high avidity and/or high affinity binding of an antibody to a specific polypeptide or carbohydrate, i.e., an epitope of a polypeptide or a carbohydrate, e.g., an L-selectin; or a CNS myelin ligand for L-selectin. For example, antibody binding to an epitope on an L-selectin polypeptide or fragment thereof is stronger than binding of the same antibody to any other epitope, particularly those which may be present in molecules in association with, or in the same sample, as the L-selectin, e.g., binds more strongly to an L-selectin than to a different polypeptide epitope so that by adjusting binding conditions the antibody binds almost exclusively to the L-selectin epitope and not to any other epitope of a polypeptide other than L-selectin, and not to any other polypeptide (or fragment) or any other polypeptide which does not comprise the epitope. Antibodies which bind specifically to a polypeptide may be capable of binding other polypeptides at a weak, yet detectable, level (e.g., 10% or less of the binding shown to the polypeptide of interest). Such weak binding, or background binding, is readily discernible from the specific antibody binding to an L-selectin, e.g. by use of appropriate controls. In general, specific antibodies bind to a given polypeptide or carbohydrate (e.g., L-selectin; a CNS myelin ligand for L-selectin) with a binding affinity of 10⁻⁷ M or more, e.g., 10⁻⁸ M or more (e.g., 10⁻⁹ M, 10⁻¹⁰ M, 10⁻¹¹ M, etc.). In general, an antibody with a binding affinity of 10⁻⁶ M or less is not useful in that it will not bind an antigen at a detectable level using conventional methodology currently used.

The term “soluble,” as used herein in the context of a soluble L-selectin or a soluble L-selectin ligand, refers to altered forms of an L-selectin or an L-selectin ligand, which altered forms lack all or part of a transmembrane domain such that the L-selectin or the L-selectin ligand is not anchored in the cell membrane.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an L-selectin antagonist” includes a plurality of such antagonists and reference to “the active agent” includes reference to one or more active agents and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides methods of treating traumatic spinal cord injury, methods of reducing cell-mediated demyelination of long descending fiber tracts or local circuits in the spinal cord following traumatic spinal cord injury, and methods for improving or restoring locomotor recovery and/or fine motor movement in an individual following spinal cord injury. The present invention further provides compositions for use in the methods. The methods generally involve administering to an individual in need thereof an effective amount of an L-selectin antagonist.

L-selectin (CD62L) is a leukocyte cell surface adhesion molecule. It is critically involved in the recruitment of leukocytes to sites of inflammation and the homing of lymphocytes into lymph nodes. L-selectin is a single-chain polypeptide that is displayed on the surface of leukocytes. The present invention is based in part on the unexpected observation that absence of L-selectin results in improved locomotor recovery in animals subjected to traumatic spinal cord injury.

Treatment Methods

The present invention provides methods of treating traumatic spinal cord injury, methods of reducing cell-mediated demyelination of long descending fiber tracts or local circuits in the spinal cord following traumatic spinal cord injury, and methods for improving, preserving, or restoring locomotor and/or fine motor movement in an individual following spinal cord injury. The methods generally involve administering to an individual in need thereof an effective amount of an L-selectin antagonist.

Spinal cord trauma can involve a tissue insult selected from abrasion, incision, contusion, puncture, compression, etc., such as can arise from traumatic contact of a foreign object with any locus of or appurtenant to the vertebral column.

Administration of an effective amount of an L-selectin antagonist to an individual who has suffered traumatic spinal cord injury reduces white matter damage, e.g., reduces cell-mediated demyelination of long descending fiber tracts or local circuits in the spinal cord of the individual. In some embodiments, an effective amount of an L-selectin antagonist is an amount that is effective to reduce cell-mediated demyelination of long descending fiber tracts or local circuits, following traumatic spinal cord injury, by at least about 2%, at least about 5%, at least about 10%, at least about 15%, at least about 20%, at least about 25%, at least about 30%, at least about 40%, at least about 50%, at least about 60%, at least about 70%, or at least about 80%, or more, when compared to the extent of demyelination in the absence of treatment with an L-selectin antagonist.

Whether cell-mediated demyelination is reduced is determined using any known method. As one example, in an experimental animal, histochemical analysis of spinal cord material using an agent such as Luxol Fast Blue, as described in the Example, is used to assess white matter. Other assays are known in the art. See, e.g., Merkler et al. (2001) J. Neuroscience 21:3665-3674. The amount of residual white matter at the lesion site is an indication of the extent of cell-mediated demyelination.

Administration of an effective amount of an L-selectin antagonist to an individual who has suffered traumatic spinal cord injury increases the rate and/or extent of locomotor recovery in the individual. In some embodiments, an effective amount of an L-selectin antagonist is an amount that increases the rate and/or extent of locomotor recovery in an individual who has suffered traumatic spinal cord injury by at least about 2%, at least about 5%, at least about 10%, at least about 15%, at least about 20%, at least about 25%, at least about 30%, at least about 40%, at least about 50%, at least about 60%, at least about 70%, or at least about 80%, or more, when compared to the rate and/or extent of locomotor recovery in the absence of treatment with an L-selectin antagonist.

The rate and extent of locomotor recovery is readily determined using any known method. Locomotor recovery can be tested using any of a variety of methods, including, but not limited to, the open field locomotor score (Basso et al. 1995, infra); the grid walk assay; the misstep withdrawal response (Gorassini et al. (1994) J. Neurophysiol. 71:603-610; and Hiebert et al. (1994) J. Neurophysiol. 71:611-622); the narrow-beam crossing assay (Metz, et al. (1998) Behav. Brain Res. 96:37-46); and the like. See, e.g., Merkler et al. (2001) J. Neuroscience 21:3665-3674. As one example, locomotor recovery is assessed using an open field testing paradigm, the Basso, Beattie, Bresnahan (BBB) Locomotor Rating Scale, that is based upon a 21 point scale originally developed in the spinal cord injured rat. Basso et al. (1995) J. Neurotrauma 12:1-21; and Basso et al. (1996) J. Neurotrauma 13:343-359. This scale assesses 10 distinct categories that range from limb movement to tail position and involve detailed observations of joint movement, stepping, and coordination. Uninjured animals exhibit a locomotor score of “21” whereas animals that exhibit complete hind limb paralysis are scored as a “0”. Animals that are moderately injured typically show recovery over time and exhibit a locomotor score of between 10 and 11 by about 6 weeks post injury.

In some embodiments, an effective amount of an L-selectin antagonist is an amount that increases the locomotor score in an individual who has suffered traumatic spinal cord injury by at least one more, at least two more, at least three more, at least four more, at least five more, at least six more, at least seven more, at least eight more, or at least nine more points in the BBB Locomotor Rating Scale, when compared to the increase in locomotor score in the absence of treatment with an L-selectin antagonist over the same time period. Thus, e.g., where the locomotor score increases by two points over a 7-day time period between 2 weeks and 3 weeks following traumatic spinal cord injury in an individual not treated with an L-selectin antagonist, an effective amount of L-selectin antagonist results in an increase in locomotor score of at least three points, at least 4 points, or more, over the same 7-day time period between 2 weeks and 3 weeks following traumatic spinal cord injury.

Administration of an effective amount of an L-selectin antagonist to an individual who has suffered traumatic spinal cord injury increases the rate and/or extent of fine motor recovery in the individual. In some embodiments, an effective amount of an L-selectin antagonist is an amount that increases the rate and/or extent of fine motor recovery in an individual who has suffered traumatic spinal cord injury by at least about 2%, at least about 5%, at least about 10%, at least about 15%, at least about 20%, at least about 25%, at least about 30%, at least about 40%, at least about 50%, at least about 60%, at least about 70%, or at least about 80%, or more, when compared to the rate and/or extent of fine motor recovery in the absence of treatment with an L-selectin antagonist.

Whether the rate and/or extent of fine motor recovery is increased is determined using any known assay method. For example, in an experimental animal, the ability to cross a grid, which assesses fine movement of the digits, can be used. The grid score represents the number of times an animal's hindlimb digits grasp a wire grid.

L-Selectin Antagonists

As used herein, the term “L-selectin antagonist” includes any agent that causes or results in decreased binding of an L-selectin to a ligand present in CNS myelin. L-selectin antagonists suitable for use in a subject method include, but are not limited to, antibodies to L-selectin; antibodies to the CNS myelin ligand of L-selectin; small molecules that inhibit binding of an L-selectin to its ligand in the CNS myelin; natural or synthetic polymers that inhibit binding of an L-selectin to its ligand in the CNS myelin, such as Fucoidin (Nasu T, Fukuda Y, Nagahira K, Kawashima H, Noguchi C, Nakanishi T. (1997) Fucoidin, a potent inhibitor of L-selectin function, reduces contact hypersensitivity reaction in mice. Immunol Lett. 59:47-51); agents that are based on actual physiological selectin ligands (e.g., naturally occurring selectin ligands) such as PSGL-1, CD34, GlyCAM-1, podocalyxin, endomucin, MADCAM-1, Sgp200, and endoglycan (see, e.g., Rosen, S. D. (2004) Ligands for L-selectin: homing, inflammation, and beyond. Annu Rev Immunol 22, 129-156), including agents that are L-selectin-binding fragments of naturally-occurring selectin ligands and that include the essential protein features and posttranslational modifications that are necessary for L-selectin binding, e.g., in the case of PSGL-1, a suitable L-selectin-binding fragment includes the extracellular region of the PSGL-1 molecule or a portion thereof fused to a heterologous peptide such as an immunoglobulin constant region (various versions of PSGL-1 with selectin binding activity are described in, e.g., Somers, W. S., Tang, J., Shaw, G. D., and Camphausen, R. T. (2000) Insights into the molecular basis of leukocyte tethering and rolling revealed by structures of P- and E-selectin bound to SLe(X) and PSGL-1. Cell 103, 467-479; Sako, D., Comess, K. M., Barone, K. M., Camphausen, R. T., Cumming, D. A., and Shaw, G. D. (1995) A sulfated peptide segment at the amino terminus of PSGL-1 is critical for P-selectin binding. Cell 83, 323-331; and Leppanen, A., Yago, T., Otto, V. I., McEver, R. P., and Cummings, R. D. (2003) Model glycosulfopeptides from PSGL-1 require tyrosine sulfation and a core-2 branched O-glycan to bind to L-selectin. J Biol Chem 278:26391-26400); an L-selectin-binding fragment of endoglycan (e.g., fragments of endoglycan with selectin binding activity are described by Fieger, C. B., Sassetti, C. M., and Rosen, S. D. (2003). Endoglycan, a member of the CD34 family, functions as a L-selectin ligand through modification with tyrosine sulfation and sialyl Lewis x. J Biol Chem 278, 27390-27398); agents that induce shedding of L-selectin from a leukocyte or other cell that mediates CNS demyelination; soluble L-selectin and ligand-binding fragments thereof; fragments of the CNS myelin ligand for L-selectin that inhibit binding of an L-selectin to its ligand in the CNS myelin; agents that reduce formation of the CNS myelin ligand for L-selectin; a polymerized glycoliposome as described in U.S. Pat. No. 6,299,897; and the like.

In some embodiments, an L-selectin antagonist competes directly or indirectly with the L-selectin CNS myelin ligand for the L-selectin binding site and, thus, reduces the proportion of L-selectin CNS myelin ligand molecules bound to the L-selectin.

L-selectin antagonists are in some embodiments synthetically produced using standard methods. See, e.g., Khadem, Carbohydrate Chemistry (Academic Press, San Diego, Calif., 1988), which is incorporated herein by reference, for synthesis of carbohydrates. Methods for synthesizing polypeptides of defined composition are well known in the art (see, Atherton et al. Solid Phase Peptide Synthesis (IRL Press, Oxford, 1989) which is incorporated herein by reference).

L-selectin antagonists are in some embodiments those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible.

In some embodiments, e.g., where an L-selectin antagonist is a polypeptide, an L-selectin antagonist is recombinantly produced using standard methods well known to those skilled in the art. For a review of standard molecular biological techniques see Sambrook et al., Molecular Cloning: A Laboratory Manual, 2d Ed. (Cold Spring Harbor Press, N.Y., 1989), which is incorporated herein by reference.

Small Molecule Antagonists

In some embodiments, an L-selectin antagonist is a small molecule. The terms “agent,” “substance,” “drug,” and “compound” are used interchangeably herein. Small molecule L-selectin antagonists include synthetic compounds, naturally-occurring compounds, fragments of naturally-occurring compounds; and the like. Small molecule L-selectin antagonists encompass numerous chemical classes, typically synthetic, semi-synthetic, or naturally-occurring inorganic or organic molecules. Small molecule L-selectin antagonists may be small organic compounds having a molecular weight of more than 50 and less than about 2,500 daltons. Small molecule L-selectin antagonists may comprise functional groups necessary for structural interaction with proteins, particularly hydrogen bonding, and may include at least an amine, carbonyl, hydroxyl or carboxyl group, and may contain at least two of the functional chemical groups. The agents may comprise cyclical carbon or heterocyclic structures and/or aromatic or polyaromatic structures substituted with one or more of the above functional groups. Small molecule L-selectin antagonists are also found among biomolecules including peptides, saccharides, fatty acids, steroids, purines, pyrimidines, derivatives, structural analogs or combinations thereof.

In some embodiments, a small molecule L-selectin antagonist is an agent that directly inhibits binding of an L-selectin to a CNS myelin ligand for L-selectin. In some embodiments, a small molecule L-selectin antagonist comprises a peptide that mimics the binding site of the L-selectin. In other embodiments, a small molecule L-selectin antagonist comprises an oligosaccharide, or a sulfated oligosaccharide, that corresponds to the CNS myelin ligand. In other embodiments, a small molecule L-selectin antagonist inhibits formation or synthesis of a CNS myelin ligand for L-selectin. In some embodiments, a small molecule L-selectin antagonist inhibits the activity of an enzyme that sulfates a CNS myelin ligand for L-selectin, where an enzyme that sulfates a CNS myelin ligand for L-selectin includes, e.g., βGal 3-O-sulfotransferase-1 (see, e.g., Honke, K., Tsuda, M., Hirahara, Y., Ishii, A., Makita, A., and Wada, Y. (1997). Molecular cloning and expression of cDNA encoding human 3′-phosphoadenylylsulfate:galactosylceramide 3′-sulfotransferase. J. Biol. Chem. 272, 4864-4868). In other embodiments, a small molecule L-selectin antagonist is an agent that induces shedding of the L-selectin from the surface of a cell that normally presents L-selectin on its surface, and that mediated spinal cord demyelination.

In some embodiments, a small molecule L-selectin antagonist is an agent that directly inhibits binding of an L-selectin to a CNS myelin ligand for L-selectin. Whether an agent inhibits binding of an L-selectin to a CNS myelin ligand for L-selectin can be determined using any known method, including, e.g., immunological assays such as an enzyme-linked immunosorbent assay (ELISA), a radioimmunoassay (RIA); a fluorescence resonance energy transfer (FRET) assay; a bioluminescence resonance energy transfer (BRET) assay; a fluorescence quenching assay; a fluorescence anisotropy assay; an immunological assay; and an assay involving binding of a detectably labeled protein to an immobilized protein; assays in which binding of L-selectin to CNS myelin is detected histochemically using spinal cord sections; and the like.

For example, a detectably labeled L-selectin is contacted with a spinal cord sample in the presence of a test agent; and the effect, if any, of the test agent on binding of the detectably labeled L-selectin to the myelin in the spinal cord is determined. The L-selectin can be labeled directly or indirectly. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures. Direct labels include enzymes that produce a detectable product (e.g., horse radish peroxidase, β-galactosidase, luciferase, alkaline phosphatase, etc.); a fluorescent protein (e.g., a green fluorescent protein; any of a variety of fluorescent and colored proteins from Anthozoan species, as described in, e.g., Matz et al. (1999) Nature Biotechnol. 17:969-973; etc.); radioisotopes; etc. Indirect labels include detectably labeled antibodies; a detectably labeled member of a specific binding pair; etc.

Immunochemical assays typically employ an antibody specific for a component of the assay (e.g., L-selectin; CNS myelin ligand for L-selectin). The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. Alternatively, the secondary antibody is conjugated to a fluorescent compound, e.g. fluorescein, rhodamine, Texas red, etc. The absence or presence of antibody binding may be determined by various methods, including flow cytometry of dissociated cells, microscopy, radiography, scintillation counting, etc.

In other embodiments, a small molecule L-selectin antagonist inhibits formation or synthesis of a CNS myelin ligand for L-selectin. Whether a small molecule agent inhibits formation of a CNS myelin ligand for L-selectin is determined using any known assay, e.g., an immunological assay employing detectably labeled antibody specific for the CNS myelin ligand of L-selectin.

In other embodiments, a small molecule L-selectin antagonist inhibits sulfation of a CNS myelin ligand for L-selectin. Whether a small molecule agent inhibits sulfation of a CNS myelin ligand for L-selectin is readily determined using an assay in which a cell that synthesizes a CNS myelin ligand for L-selectin is cultured in the presence of a radiolabelled sulfate, and the effect, if any, of a test agent on incorporation of the radiolabelled sulfate into the CNS myelin ligand for L-selectin is determined.

In other embodiments, a small molecule L-selectin antagonist is an agent that induces shedding of the L-selectin from the surface of a cell that normally presents L-selectin on its surface, and that mediates spinal cord demyelination. Of particular interest is an agent that induces shedding of L-selectin from the surface of a cell that mediates demyelination. Whether an agent induces shedding of L-selectin from the surface of a cell that mediates demyelination is determined by monitoring release of detectably labeled L-selectin from the surface of cells of interest. For example, cells are cultured in the presence (or, as a control, in the absence) of a test agent; and cells are then contacted with a detectably labeled antibody to L-selectin. Labeling of cells is readily detected using, e.g., fluorescence activated cell sorting. See, e.g., U.S. Pat. No. 6,498,189. Alternatively, an ELISA assay for soluble L-selectin can be used. See, e.g., U.S. Pat. No. 6,498,189.

Cells that normally present L-selectin on their surface, and that mediate spinal cord demyelination, include inflammatory cells such as lymphocytes, monocytes/macrophages, eosinophils, basophils, neutrophils, and microglia.

Agents that induce shedding of L-selectin include, but are not limited to, glucocorticoids; annexin 1; non-steroidal anti-inflammatory agents (NSAIDs); promoters of TNF-α converting enzyme activity (TACE) (see, e.g., U.S. Pat. No. 6,632,667; U.S. Pat. No. 5,629,285), including inhibitors of calmodulin (Kahn, J., Walcheck, B., Migaki, G. I., Jutila, M. A., and Kishimoto, T. K. (1998). Calmodulin regulates L-selectin adhesion molecule expression and function through a protease-dependent mechanism. Cell 92, 809-818); agents that promote clustering of L-selectin at the cell surface, e.g., multivalent ligands dubbed “neoglycopolymers” which present multiple copies of saccharide epitopes on an extended backbone (see, e.g., Gordon et al. (1998) Nature 392:30-31); and the like.

Glucocorticoids include, but are not limited to, prednisone; prednisolone; methyl prednisolone; dexamethasone; beta metasone dehydroepiandrosterone; 9a-fluorocortisol; prednisone; aetiocholanolone; 2-methylcortisol; pregnanediol; deoxycorticosterone; cortisone; hydrocortisone (cortisol); 6a-methylprednisolone; triamcinolone; a 21-aminosteroid; and the like. NSAIDs, include, but are not limited to, 1) the oxicams, such as piroxicam, isoxicam, tenoxicam, and sudoxicam; 2) the salicylates, such as aspirin, disalcid, benorylate, trilisate, safapryn, solprin, diflunisal, and fendosal; 3) the acetic acid derivatives, such as diclofenac, fenclofenac, indomethacin, sulindac, tolmetin, isoxepac, furofenac, tiopinac, zidometacin, acematacin, fentiazac, zomepiract, clidanac, oxepinac, and felbinac; 4) the fenamates, such as mefenamic, meclofenamic, flufenamic, niflumic, and tolfenamic acids; 5) the propionic acid derivatives, such as ibuprofen, naproxen, benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and 6) the pyrazoles, such as phenylbutazone, oxyphenbutazone, feprazone, azapropazone, and trimethazone, mixtures of these non-steroidal anti-inflammatory agents may also be employed, as well as the pharmaceutically-acceptable salts and esters of these agents.

TACE activators include any compound which promotes the activity or the expression (i.e., the synthesis) of TACE. Examples of such compounds include inhibitors of calmodulin (Kahn, J., Walcheck, B., Migaki, G. I., Jutila, M. A., and Kishimoto, T. K. (1998). Calmodulin regulates L-selectin adhesion molecule expression and function through a protease-dependent mechanism. Cell 92, 809-818.)

Peptide Antagonists

In some embodiments, an L-selectin antagonist is an agent that directly inhibits binding of an L-selectin to a CNS myelin ligand for L-selectin, which agent comprises a peptide fragment of L-selectin, or a derivative or variant of such a peptide. Suitable peptides are discussed in, e.g., Briggs, J. B., Larsen, R. A., Harris, R. B., Sekar, K. V., and Macher, B. A. (1996). Structure/activity studies of anti-inflammatory peptides based on a conserved peptide region of the lectin domain of E-, L- and P-selectin. Glycobiology 6, 831-836. In general, suitable peptide antagonist comprise from about 5 to about 50 contiguous amino acids of the extracellular portion of an L-selectin. For example, a suitable peptide antagonist comprises from about 5 to about 50 contiguous amino acids of amino acids 39-332 of SEQ ID NO:1, e.g, from about 5 to about 10, from about 10 to about 15, from about 15 to about 20, from about 20 to about 25, from about 25 to about 30, from about 30 to about 35, from about 35 to about 40, from about 40 to about 45, or from about 45 to about 50 contiguous amino acids of amino acids 39-332 of SEQ ID NO:1.

In some embodiments, a peptide antagonist comprises a peptide fragment of the extracellular region of an L-selectin linked to another moiety, such as a carrier or a functional moiety. In some embodiments, a peptide antagonist is linked to (e.g., covalently linked; or non-covalently linked) to a heterologous peptide (e.g., a peptide other than an L-selectin peptide); a lipid; a carbohydrate; and the like.

The peptide antagonist can be used in the form of the free peptide or a pharmaceutically acceptable salt. Amine salts can be prepared by mixing the peptide with an acid according to known methods. Suitable acids include inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalenesulfonic acid, and sulfanilic acid.

Guidance for the selection of peptide inhibitors of L-selectin that are suitable for use herein is found in U.S. Pat. No. 6,111,065.

For example, in some embodiments, suitable peptide inhibitors include peptides of the formula R¹-X-Gly-Ile-Trp-Y-R² wherein X and Y are linear chains of from 0 to 16 amino acids; R¹ is H (signifying a free N-terminal primary amino group), formyl, lower alkyl, aryl, lower alkanoy, aroyl, alkyloxycarbonyl or aryloxycarbonyl; and R² is OH (signifying a free C-terminal carboxyl group), lower alkyl or aryl esters, or NR³R⁴ where NR³R⁴ are each selected independently from H, lower alkyl or aryl. Gly-Ile-Trp corresponds to amino acids 96-98 of SEQ ID NO:1. In some embodiments, a suitable peptide antagonist comprises X-Gly-Ile-Trp-Y, wherein X is from 0 to 16 amino acids of amino acids 80-95 of SEQ ID NO:1; and Y is from 0 to 16 amino acids of amino acids 99-114 of SEQ ID NO:1.

The peptides can generally be prepared following known techniques, as described for example in the cited publications, the teachings of which are specifically incorporated herein. In some embodiments, the peptides are prepared following the solid-phase synthetic technique initially described by Merrifield in J. Amer. Chem. Soc., 85, 2149-2154 (1963). Other techniques may be found, for example, in M. Bodanszky, et al., Peptide Synthesis, second edition, (John Wiley & Sons, 1976), as well as in other reference works known to those skilled in the art.

The peptides can also be prepared using standard genetic engineering techniques known to those skilled in the art. For example, the peptide can be produced by inserting nucleic acid encoding the peptide into an expression vector, expressing the DNA, and translating the RNA into the peptide in the presence of the required amino acids. The peptide is then purified using chromatographic or electrophoretic techniques, or by means of a carrier protein which can be fused to, and subsequently cleaved from, the peptide by inserting into the expression vector in phase with the peptide encoding sequence a nucleic acid sequence encoding the carrier protein. The fusion protein-peptide may be isolated using chromatographic, electrophoretic or immunological techniques (such as binding to a resin via an antibody to the carrier protein). The peptide can be cleaved using chemical methodology or enzymatically, as by, for example, hydrolases.

Antibodies

In some embodiments, the L-selectin antagonist is an antibody. In some embodiments, the L-selectin antagonist is an antibody specific for L-selectin. In other embodiments, the L-selectin antagonist is an antibody specific for a CNS myelin ligand for L-selectin.

Antibodies that specifically bind L-selectin and that are suitable for use in a subject method are antibodies that inhibit binding of an L-selectin to a CNS myelin ligand for L-selectin. Suitable antibodies include those discussed in U.S. Pat. No. 5,227,369. Suitable antibodies include, but are not limited to, antibodies of various isotypes (e.g., IgG1, IgG3 and IgG4); polyclonal antibodies; monoclonal antibodies produced by any means; humanized antibodies; chimeric antibodies; single-chain antibodies; antibody fragments such as Fv, F(ab′)₂, Fab′, Fab, and the like; and the like, provided that the antibody is capable of specific binding to an L-selectin and inhibiting binding of the L-selectin to a CNS myelin ligand for L-selectin. Suitable antibodies typically bind to an L-selectin with an affinity of at least about 10⁻⁸ M, at least about 10⁻⁹ M, at least about 10⁻¹⁰ M, or greater.

Mouse antibodies specific for L-selectin have been described, including, e.g., mouse DREG-55, mouse DREG-56 and mouse DREG-200, which antibodies bind to human L-selectin (Kishimoto et al., Proc. Natl. Acad. Sci. USA 87:2244 (1990); TQ-1; and the LAM series of antibodies (Spertini, O., Kansas, G. S., Reimann, K. A., Mackay, C. R., and Tedder, T. F. (1991). Function and evolutionary conservation of distinct epitopes on the leukocyte adhesion molecule-1 (TQ-1, Leu-8) that regulate leukocyte migration. J Immunol 147(3), 942-949; and Steeber, D. A., Engel, P., Miller, A. S., Sheetz, M. P., and Tedder, T. F. (1997). Ligation of L-selectin through conserved regions within the lectin domain activates signal transduction pathways and integrin function in human, mouse, and rat leukocytes. J Immunol 159, 952-963.). Humanized antibodies to L-selectin have been described in U.S. Pat. No. 6,210,671; such humanized anti-L-selectin antibodies are suitable for use in a subject method.

In other embodiments, the L-selectin antagonist is an antibody specific for a CNS myelin ligand for L-selectin. Antibodies that specifically bind a CNS myelin ligand for L-selectin and that are suitable for use in a subject method are antibodies that inhibit binding of an L-selectin to a CNS myelin ligand for L-selectin. Suitable antibodies include, but are not limited to, antibodies of various isotypes (e.g., IgG1, IgG3 and IgG4); polyclonal antibodies; monoclonal antibodies produced by any means; humanized antibodies; chimeric antibodies; single-chain antibodies; antibody fragments such as Fv, F(ab′)₂, Fab′, Fab, and the like; and the like, provided that the antibody is capable of specific binding to a CNS myelin ligand for L-selectin and inhibiting binding of the L-selectin to a CNS myelin ligand for L-selectin. Suitable antibodies typically bind to a CNS myelin ligand for L-selectin with an affinity of at least about 10⁻⁸ M, at least about 10⁻⁹ M, at least about 10⁻¹⁰ M, or greater.

For preparation of polyclonal antibodies, the first step is immunization of the host animal with the target antigen (e.g., protein or carbohydrate), where the target antigen will preferably be in substantially pure form, comprising less than about 1% contaminant. The immunogen may comprise the complete target protein, fragments or derivatives thereof. To increase the immune response of the host animal, the target protein may be combined with an adjuvant, where suitable adjuvants include alum, dextran, sulfate, large polymeric anions, oil and water emulsions, e.g. Freund's adjuvant, Freund's complete adjuvant, and the like. The target antigen may also be conjugated to synthetic carrier proteins or synthetic antigens. A variety of hosts may be immunized to produce the polyclonal antibodies. Such hosts include rabbits, guinea pigs, rodents, e.g. mice, rats, sheep, goats, and the like. The target antigen is administered to the host, usually intradermally, with an initial dosage followed by one or more, usually at least two, additional booster dosages. Following immunization, the blood from the host will be collected, followed by separation of the serum from the blood cells. The Ig present in the resultant antiserum may be further fractionated using known methods, such as ammonium salt fractionation, DEAE chromatography, and the like.

Monoclonal antibodies are produced by conventional techniques. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. Suitable animals for production of monoclonal antibodies to the human protein include mouse, rat, hamster, etc. To raise antibodies against the mouse protein, the animal will generally be a hamster, guinea pig, rabbit, etc. The antibody may be purified from the hybridoma cell supernatants or ascites fluid by conventional techniques, e.g. affinity chromatography using protein bound to an insoluble support, protein A sepharose, etc.

The antibody may be produced as a single chain, instead of the normal multimeric structure. Single chain antibodies are described in Jost et al. (1994) J. Biol. Chem. 269:26267-73, and others. DNA sequences encoding the variable region of the heavy chain and the variable region of the light chain are ligated to a spacer encoding at least about 4 amino acids of small neutral amino acids, including glycine and/or serine. The protein encoded by this-fusion allows assembly of a functional variable region that retains the specificity and affinity of the original antibody.

Also of interest in certain embodiments are humanized antibodies. Methods of humanizing antibodies are known in the art. The humanized antibody may be the product of an animal having transgenic human immunoglobulin constant region genes (see for example International Patent Applications WO 90/10077 and WO 90/04036). Alternatively, the antibody of interest may be engineered by recombinant DNA techniques to substitute the CH1, CH2, CH3, hinge domains, and/or the framework domain with the corresponding human sequence (see WO 92/02190).

The use of Ig cDNA for construction of chimeric immunoglobulin genes is known in the art (Liu et al. (1987) Proc. Natl. Acad. Sci. USA 84:3439 and (1987) J. Immunol. 139:3521). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al. (1991) Sequences of Proteins of Immunological Interest, N.I.H. publication no. 91-3242. Human C region genes are readily available from known clones. The choice of isotype will be guided by the desired effector functions, such as complement fixation, or activity in antibody-dependent cellular cytotoxicity. Preferred isotypes are IgG1, IgG3 and IgG4. Either of the human light chain constant regions, kappa or lambda, may be used. The chimeric, humanized antibody is then expressed by conventional methods.

Antibody fragments, such as Fv, F(ab′)₂ and Fab may be prepared by cleavage of the intact protein, e.g. by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Consensus sequences of H and L J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g. SV-40 early promoter, (Okayama et al. (1983) Mol. Cell. Bio. 3:280), Rous sarcoma virus LTR (Gorman et al. (1982) P.N.A.S. 79:6777), and moloney murine leukemia virus LTR (Grosschedl et al. (1985) Cell 41:885); native Ig promoters, etc.

Soluble L-Selectin

In some embodiments, an L-selectin antagonist is a soluble L-selectin. A soluble L-selectin is a fragment of an L-selectin; is not membrane bound; and competes for membrane-bound (e.g., cell surface) L-selectin for binding to CNS myelin ligand for L-selectin. A soluble L-selectin typically comprises at least a portion of the extracellular region of L-selectin, and will generally comprise at least the portion of the extracellular region of L-selectin that binds a CNS myelin ligand. A soluble L-selectin typically lacks the transmembrane and cytoplasmic portions of L-selectin.

A soluble L-selectin will in some embodiments be recombinantly produced using standard methods well known to those skilled in the art. In addition, using standard recombinant DNA techniques, mutations can be induced to obtain proteins with altered amino acid sequences. Typically, substitutions, deletions or additions are introduced which provide desired characteristics.

In some embodiments, an L-selectin antagonist is a fusion protein comprising at least the CNS myelin ligand-binding portion of the extracellular region of L-selectin;

and a heterologous polypeptide (a “fusion partner”). Suitable fusion partners include, but are not limited to, an immunoglobulin constant region; hemagglutinin; an epitope such as FLAG, and the like; proteins that provide for a detectable signal, including, but not limited to, fluorescent proteins, enzymes (e.g., β-galactosidase, luciferase, horse radish peroxidase, etc.), avidin, and the like; polypeptides that facilitate purification or isolation of the fusion protein, e.g., metal ion binding polypeptides such as poly-histidine (e.g., 6His), glutathione-S-transferase, and the like; polypeptides that provide for increased solubility; and polypeptides that provide for increased stability.

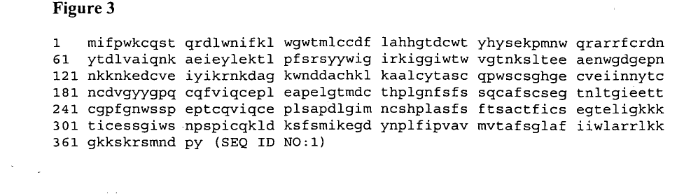

The amino acid sequences of L-selectins (CD62L) are known and are publicly available in, e.g., public databases such as GenBank; journal articles; and issued patents. For example, amino acid sequences of L-selectins are found under GenBank Accession Nos. P18337 (mouse L-selectin); and NP_{—}000646 (human L-selectin); and Siegelman and Weissman (1989) Proc. Natl. Acad. Sci. USA 86:5562-5566. An amino acid sequence of human L-selectin is provide in FIG. 3 (SEQ ID NO:1). The extracellular region is amino acids 39-332 of SEQ ID NO:1. Amino acids 1-38 are not included in the mature protein. The transmembrane region is amino acids 333-355 of SEQ ID NO:1. Thus, in some embodiments, a soluble L-selectin ligand comprises from about 15 to about 294 contiguous amino acids of amino acids 39-332 of SEQ ID NO:1, e.g., from about 15 to about 20, from about 20 to about 30, from about 30 to about 40, from about 40 to about 50, from about 50 to about 60, from about 60 to about 70, from about 70 to about 80, from about 80 to about 100, from about 100 to about 125, from about 125 to about 150, from about 150 to about 175, from about 175 to about 200, from about 200 to about 225, from about 225 to about 250, or from about 250 to about 294 contiguous amino acids of amino acids 39-332 of SEQ ID NO:1; or a variant comprising conservative amino acid sequence changes thereof.

In some embodiments, an L-selectin antagonist is a soluble L-selectin ligand. Soluble L-selectin ligands that are suitable for use in a subject method include, but are not limited to, L-selectin-binding fragments of naturally-occurring selectin ligands, e.g., fragments of naturally-occurring selectin ligands that include the essential protein features and posttranslational modifications that are necessary for L-selectin binding. Suitable soluble L-selectin ligands include, but are not limited to, an L-selectin binding extracellular region of the PSGL-1 molecule; an L-selectin-binding fragment of endoglycan; and the like. Also suitable for use as L-selectin antagonists is a fusion protein comprising a soluble L-selectin ligand fused to a heterologous peptide, such as an immunoglobulin constant region. Also included are soluble L-selectin ligands comprising conservative amino acid changes, relative to a naturally-occurring L-selectin ligand. In some embodiments, a soluble L-selectin ligand is a sulfatide, or an L-selectin-binding fragment of a sulfatide.

Dosages, Formulations, and Routes of Administration

An active agent (e.g., an L-selectin antagonist; a second therapeutic agent, etc.; also referred to herein as a “drug” or a “therapeutic agent”) is administered to individuals in a formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20^{th} edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In the subject methods, an active agent (e.g., an L-selectin antagonist; a second therapeutic agent, etc.) may be administered to the host using any convenient means capable of resulting in the desired therapeutic effect. Thus, the agents can be incorporated into a variety of formulations for therapeutic administration. More particularly, an active agent (e.g., an L-selectin antagonist; a second therapeutic agent, etc.) can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of an active agent(s) can be achieved in various ways, including oral, buccal, rectal, parenteral, intraperitoneal, intradermal, intrathecal, intraspinal, intracistemal, intracapsular, subcutaneous, intravenous, intramuscular, transdermal, intratracheal, etc., administration. In some embodiments, e.g., where two different agents are administered, two different routes of administration are used. Where the active agent is to be provided parenterally, such as by intravenous, subcutaneous, ophthalmic, intraperitoneal, intramuscular, buccal, rectal, vaginal, intraorbital, intracerebral, intracranial, intraspinal, intraventricular, intrathecal, intracisternal, intracapsular, intranasal or by aerosol administration, the agent typically comprises part of an aqueous or physiologically compatible fluid suspension or solution.

A liquid is in some embodiments the dosage form that is used for intravenous, intrathecal, intraspinal, intraventricular, or intramedullary administration of an active agent for treating spinal cord injuries. For preparing liquids, solvents can be used, as exemplified by purified water, physiological saline, alcohols such as ethanol, propylene glycol, glycerin and polyethylene glycol, and triacetin. The thus prepared liquids may be used as dilutions with a lactated Ringer's solution, a maintaining solution, a postoperative recovery fluid, a solution for supplying water to compensate for dehydration, physiological saline for use in dripping. The preparations may further be admixed with adjuvants such as antiseptics, moistening agents, emulsifiers, dispersing agents and stabilizers. Suspensions are another exemplary dosage form to be administered.

In some embodiments, an active agent (e.g., an L-selectin antagonist; a second therapeutic agent, etc.) is administered intrathecally, including, e.g., administration into a cerebral ventricle, administration into the lumbar area, and administration into the cisterna magna; or by an intraspinal route. For specific delivery within the CNS intrathecal delivery can be used with, for example, an Ommaya reservoir. U.S. Pat. No. 5,455,044 provides for use of a dispersion system for CNS delivery or see U.S. Pat. No. 5,558,852 for a discussion of CNS delivery.

As used herein, the term “intrathecal administration” includes delivering an active agent directly into the cerebrospinal fluid of a subject, by techniques including lateral cerebroventricular injection through a burrhole or cisternal or lumbar puncture or the like (e.g., as described in Lazorthes et al. Advances in Drug Delivery Systems and Applications in Neurosurgery, 143-192 and Omaya et al., Cancer Drug Delivery, 1: 169-179, the contents of which are incorporated herein by reference). The term “lumbar region” includes the area between the third and fourth lumbar (lower back) vertebrae. The term “cisterna magna” includes the area where the skull ends and the spinal cord begins at the back of the head. The term “cerebral ventricle” includes the cavities in the brain that are continuous with the central canal of the spinal cord. Administration of an active agent to any of the above mentioned sites can be achieved by direct injection of the active agent or by the use of infusion pumps. For injection, the active agent can be formulated in liquid solutions, preferably in physiologically compatible buffers such as Hank's solution or Ringer's solution. In addition, the active agent may be formulated in solid form and re-dissolved or suspended immediately prior to use. Lyophilized forms are also included. The injection can be, for example, in the form of a bolus injection or continuous infusion (e.g., using infusion pumps) of the active agent.

Subcutaneous administration of an active agent (e.g., an L-selectin antagonist; a second therapeutic agent, etc.) can be accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of an active agent to a patient through the port is referred to herein as “a subcutaneous injection port delivery system.” In some embodiments, subcutaneous administration is achieved by a combination of devices, e.g., bolus delivery by needle and syringe, followed by delivery using a continuous delivery system.

In some embodiments, an active agent (e.g., an L-selectin antagonist; a second therapeutic agent, etc.) is delivered by a continuous delivery system. The terms “continuous delivery system,” “controlled delivery system,” and “controlled drug delivery device,” are used interchangeably to refer to controlled drug delivery devices, and encompass pumps in combination with catheters, injection devices, and the like, a wide variety of which are known in the art.

Mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, the present methods of drug delivery can be accomplished using any of a variety of refillable, pump systems. Pumps provide consistent, controlled release over time. Typically, the agent is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to a subdermal, subcutaneous, intramuscular, or other suitable site within a subject's body. Subcutaneous implantation sites are generally preferred because of convenience in implantation and removal of the drug delivery device.

Drug release devices suitable for use in the invention may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic-system, etc.

Drug release devices based upon a mechanical or electromechanical infusion pump can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, non-exchangeable pump systems. Pumps and other convective systems are generally preferred due to their generally more consistent, controlled release over time. Osmotic pumps are particularly preferred due to their combined advantages of more consistent controlled release and relatively small size (see, e.g., PCT published application no. WO 97/27840 and U.S. Pat. Nos. 5,985,305 and 5,728,396)). Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like.

In some embodiments, the drug delivery device is an implantable device. The drug delivery device can be implanted at any suitable implantation site using methods and devices well known in the art. As noted infra, an implantation site is a site within the body of a subject at which a drug delivery device is introduced and positioned. Implantation sites include, but are not necessarily limited to a subdermal, subcutaneous, intramuscular, intraspinal, or other suitable site within a subject's body.

In some embodiments, a therapeutic agent is delivered using an implantable drug delivery system, e.g., a system that is programmable to provide for administration of a therapeutic agent. Exemplary programmable, implantable systems include implantable infusion pumps. Exemplary implantable infusion pumps, or devices useful in connection with such pumps, are described in, for example, U.S. Pat. Nos. 4,350,155; 5,443,450; 5,814,019; 5,976,109; 6,017,328; 6,171,276; 6,241,704; 6,464,687; 6,475,180; and 6,512,954. A further exemplary device that can be adapted for the present invention is the SynchroMed® infusion pump (Medtronic).

In pharmaceutical dosage forms, an active agent (e.g., an L-selectin antagonist; a second therapeutic agent, etc.) may be administered in the form of their pharmaceutically acceptable salts, or they may also be used alone or in appropriate association, as well as in combination, with other pharmaceutically active compounds. The following methods and excipients are merely exemplary and are in no way limiting.

An active agent (e.g., an L-selectin antagonist; a second therapeutic agent, etc.) can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

For oral preparations, an active agent (e.g., an L-selectin antagonist; a second therapeutic agent, etc.) is formulated alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives, and flavoring agents.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more active agents. Similarly, unit dosage forms for injection or intravenous administration may comprise the agent(s) in a composition as a solution in sterile water, normal saline or another pharmaceutically acceptable carrier.

Dosages

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the a unit dosage forms of the present invention depend on the particular compound employed and the effect to be achieved, and the pharmacodynamics associated with each compound in the host.

Generally, unit dosage forms of an L-selectin antagonist range from about 1 μg to about 500 mg, e.g., from about 1 μg to about 5 μg, from about 5 μg to about 10 μg, from about 10 μg to about 25 μg, from about 25 μg to about 50 μg, from about 50 μg to about 75 μg, from about 75 μg to about 100 μg, from about 100 μg to about 125 μg, from about 125 μg to about 150 μg, from about 150 μg to about 200 μg, from about 200 μg to about 225 μg, from about 225 μg to about 250 μg, from about 250 μg to about 275 μg, from about 275 μg to about 300 μg, from about 300 μg to about 400 μg, from about 400 μg to about 500 μg, from about 500 μg to about 600 μg, from about 600 μg to about 700 μg, from about 700 μg to about 800 μg, from about 800 μg to about 900 μg, from about 900 μg to about 1000 μg, from about 1 mg to about 100 mg, from about 100 mg to about 200 mg, or from about 200 mg to about 500 mg.

An L-selectin antagonist can be administered twice daily, daily, every other day, once a week, twice a week, three times a week, every other week, three times per month, or once monthly, or substantially continuously or continuously.

An L-selectin antagonist is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time.

In some embodiments, an L-selectin antagonist is administered within about 1 minute to about 48 hours of a traumatic spinal cord injury, e.g., an L-selectin antagonist is administered within from about 1 minute to about 5 minutes, from about 5 minutes to about 10 minutes, from about 10 minutes to about 15 minutes, from about 15 minutes to about 30 minutes, from about 30 minutes to about 45 minutes, from about 45. minutes to about 60 minutes, from about 1 hour to about 2 hours, from about 2 hours to about 4 hours, from about 4 hours to about 6 hours, from about 6 hours to about 8 hours, from about 8 hours to about 12 hours, from about 12 hours to about 16 hours, from about 16 hours to about 24 hours, from about 24 hours to about 36 hours, or from about 36 hours to about 48 hours, following a traumatic spinal cord injury.

In some embodiments, an L-selectin antagonist is administered at or near the site of spinal cord injury. For example, in some embodiments, the route of administration of an L-selectin antagonist is selected from an intrathecal, an intraspinal, an intracisternal, or an intraventricular route of administration.

Pharmaceutical Compositions

The present invention provides pharmaceutical compositions in a unit dosage form for treating or ameliorating neurological disorders that accompany traumatic spinal cord injuries. A subject pharmaceutical composition generally comprises: a) a therapeutically effective amount of an L-selectin antagonist; b) at least one additional agent (e.g., a second L-selectin antagonist that is different from the L-selectin antagonist in (a); or an agent other than an L-selectin antagonist) that is effective for the amelioration of neurological symptoms associated with spinal cord injuries; and c) a pharmaceutically acceptable carrier or diluent.

In some embodiments, a subject pharmaceutical composition comprises: a) a therapeutically effective amount of a first L-selectin antagonist that is effective for the amelioration of neurological symptoms associated with spinal cord injuries; b) a therapeutically effective amount of a second L-selectin antagonist that is effective for the amelioration of neurological symptoms associated with spinal cord injuries; and c) a pharmaceutically acceptable carrier or diluent.

In some embodiments, a subject pharmaceutical composition comprises: a) a therapeutically effective amount of an L-selectin antagonist that is effective for the amelioration of neurological symptoms associated with spinal cord injuries; b) at least one additional agent other than an L-selectin antagonist) that is effective for the amelioration of neurological symptoms associated with spinal cord injuries; and c) a pharmaceutically acceptable carrier or diluent.

Suitable second additional therapeutic agents that are effective for the amelioration of neurological symptoms associated with spinal cord injuries include, but are not limited to, a steroid; an antioxidant; a ganglioside; a calcium channel blocker; an inhibitor of lipid peroxidation; a blocker of caspase activation; a glutamate receptor antagonist; an agent that interferes with matrix proteoglycans, e.g., chondroitin sulfate, which agents include, e.g., chondroitinase A and/or B and/or C; an agent that inhibits chondroitin sulfate biosynthesis; and the like.

Suitable antioxidants include, but are not limited to, ascorbic acid; ascorbyl palmitate; butylated hydroxytoluene; butylated hydroxyanisole; propyl gallate; a tocopherol; lipoic acid (including a lipoic acid derivative (see, e.g., U.S. Pat. No. 6,605,637) and an optical isomer of lipoic acid (see, e.g., U.S. Pat. No. 6,664,287); N-acetyl cysteine; a carotenoid; pyrrolidine dithiocarbamate; a vitamin E derivative (see, e.g., U.S. Pat. No. 6,387,882); Coenzyme Q10; Ebselen; porphyrin catalytic antioxidant manganese (III) meso-tetrakis (N-ethylpyridinium-2-yl) porphyrin; (MnTE-2-PyP (5+)); disodium 4-[(tert-butylimino) methyl] benzene-1,3-disulfonate N-oxide (NXY-059); N:-t-butyl-phenylnitrone; Tirilazadand the like.

Suitable gangliosides include, but are not limited to, GM, (see, e.g., U.S. Pat. No. 6,620,793).

Suitable calcium channel-blockers include, but are not limited to, nifedipine. (Procardia); verapamil (Calan); dihydropyridines such as nicardipine, nimodipine, and the like; benzothiazepines such as dilitazem; amiloride; amlodipine; felodipine; isradipine; diarylaminopropylamine ethers such as bepridil; and benzimidole-substituted tetralines such as mibefradil; and the like.

Suitable glutamate receptor antagonists include, but are not limited to, an α-amino-3-hydroxy-5-methyl-4-isoxazole propionic acid (AMPA) receptor antagonist such as GYKI 52466, NBQX, YM90K, YN872, ZK-200775, or MPQX; D-AP5 (D(−)-2-amino-5-phosphonopentanoate); CGS19755 (4-phosphonomethyl-2-piperidine carboxylic acid); CGP37849 (D,L-(E)-2-amino-4-methylphosphono-3-pentanoic acid); LY233053 (cis-(.+-.)-4-(2H-tetrazol-5-yl)methyl-piperidine-2-carboxyl acid); AIDA (1-aminoindan-1,5(RS)-dicarboxylic acid); (s)-(+)-CBPG ((S)-(+)-2-(3′-carboxybicyclo(1.1.1.)pentyl)glycine); CPCCOEt (cyclopropan(b)chromen-1a-carboxylate); EGLU ((s)-(α)-ethylglutamate); LY307452 (2s,4s-2-amino-4-(4,4-diphenylbut-1-yl)pentan-1,5-dioc acid); LY341495 (2s-2-amino-2-(1s,2s-2-carboxy-cyclopropan-1-yl)-3-(xanth-9-yl)propanoic acid); PCCG-4 (2s,1′s,2′s,3′R)-2-(2′-carboxy-3′-phenylcyclopropyl)glycine); 4-CPG (4-carboxyphenylglycine); memantine; amantadine; a 2,3-quinoxalinedione as described in U.S. Pat. No. 6,172,065; an N-methyl-D-aspartate (NMDA) receptor antagonist (e.g., an NMDA receptor antagonist as described in U.S. Pat. No. 6,649,605); a kainate receptor antagonist; and the like.

In some embodiments, a subject composition comprises: a) a therapeutically effective amount of an L-selectin antagonist; b) a steroid effective for the amelioration of neurological symptoms associated with spinal cord injuries; and c) a pharmaceutically acceptable carrier or diluent. In some embodiments, the steroid is dexamethasone. In other embodiments, the steroid is methylprednisolone. Other suitable steroids are listed below.

In some embodiments, a subject composition comprises: a) a therapeutically effective amount of an L-selectin antagonist; b) an antioxidant effective for the amelioration of neurological symptoms associated with spinal cord injuries; and c) a pharmaceutically acceptable carrier or diluent.

In some embodiments, a subject composition comprises: a) a therapeutically effective amount of an L-selectin antagonist; b) a ganglioside effective for the amelioration of neurological symptoms associated with spinal cord injuries; and c) a pharmaceutically acceptable carrier or diluent.

In some embodiments, a subject composition comprises: a) a therapeutically effective amount of an L-selectin antagonist; b) a calcium channel blocker effective for the amelioration of neurological symptoms associated with spinal cord injuries; and c) a pharmaceutically acceptable carrier or diluent.

In some embodiments, a subject composition comprises: a) a therapeutically effective amount of an L-selectin antagonist; b) an inhibitor of lipid peroxidation effective for the amelioration of neurological symptoms associated with spinal cord injuries; and c) a pharmaceutically acceptable carrier or diluent.

In some embodiments, a subject composition comprises: a) a therapeutically effective amount of an L-selectin antagonist; b) a blocker of caspase activation effective for the amelioration of neurological symptoms associated with spinal cord injuries; and c) a pharmaceutically acceptable carrier or diluent.

In some embodiments, a subject composition comprises: a) a therapeutically effective amount of an L-selectin antagonist; b) a glutamate receptor antagonist effective for the amelioration of neurological symptoms associated with spinal cord injuries; and c) a pharmaceutically acceptable carrier or diluent.

In some embodiments, a subject composition comprises: a) a therapeutically effective amount of an L-selectin antagonist; b) a chondroitinase effective for the amelioration of neurological symptoms associated with spinal cord injuries; and c) a pharmaceutically acceptable carrier or diluent.

In some embodiments, a subject composition comprises: a) a therapeutically effective amount of an L-selectin antagonist; b) an antioxidant effective for the amelioration of neurological symptoms associated with spinal cord injuries; c) a steroid; and d) a pharmaceutically acceptable carrier or diluent.

In some embodiments, a subject composition comprises: a) a therapeutically effective amount of an L-selectin antagonist; b) a ganglioside effective for the amelioration of neurological symptoms associated with spinal cord injuries; c) a steroid; and d) a pharmaceutically acceptable carrier or diluent.

In some embodiments, a subject composition comprises: a) a therapeutically effective amount of an L-selectin antagonist; b) a calcium channel blocker effective for the amelioration of neurological symptoms associated with spinal cord injuries; c) a steroid; and d) a pharmaceutically acceptable carrier or diluent.

In some embodiments, a subject composition comprises: a) a therapeutically effective amount of an L-selectin antagonist; b) an inhibitor of lipid peroxidation effective for the amelioration of neurological symptoms associated with spinal cord injuries; c) a steroid; and d) a pharmaceutically acceptable carrier or diluent.

In some embodiments, a subject composition comprises: a) a therapeutically effective amount of an L-selectin antagonist; b) a blocker of caspase activation effective for the amelioration of neurological symptoms associated with spinal cord injuries; c) a steroid; and d) a pharmaceutically acceptable carrier or diluent.

In some embodiments, a subject composition comprises: a) a therapeutically effective amount of an L-selectin antagonist; b) a glutamate receptor antagonist effective for the amelioration of neurological symptoms associated with spinal cord injuries; c) a steroid; and d) a pharmaceutically acceptable carrier or diluent.

In some embodiments, a subject composition comprises: a) a therapeutically effective amount of an L-selectin antagonist; b) a chondroitinase effective for the amelioration of neurological symptoms associated with spinal cord injuries; c) a steroid; and d) a pharmaceutically acceptable carrier or diluent.

Combination Therapies

In some embodiments, a subject method includes administering to an individual in need thereof an effective amount of an L-selectin antagonist and an effective amount of at least a second agent that is therapeutic in the treatment of traumatic spinal cord injury.

In some embodiments, an L-selectin antagonist and a second therapeutic agent are administered in the same formulation (e.g., the L-selectin antagonist and the second therapeutic agent are co-formulated). In other embodiments, an L-selectin antagonist and a second therapeutic agent are administered in separate formulations; and are administered simultaneously. In other embodiments, an L-selectin antagonist and a second therapeutic agent are administered in separate formulations; and the L-selectin antagonist is administered within about 1 minute to about 1 hour of administration of the second therapeutic agent.

In some embodiments, two different L-selectin antagonists are administered. The following are non-limiting examples. In some embodiments, an L-selectin antagonist that is an antibody specific for a CNS myelin L-selectin ligand; and an L-selectin antagonist that is an antibody specific for L-selectin are administered. In other embodiments, an L-selectin antagonist that is a soluble form of L-selectin; and an L-selectin antagonist that is an antibody specific for L-selectin are administered. In other embodiments, an L-selectin antagonist that is a soluble form of an L-selectin CNS myelin ligand; and an L-selectin antagonist that is a soluble form of L-selectin are administered. In other embodiments, a compound that induces shedding of L-selectin ligand from a cell that mediates CNS demyelination; and an L-selectin antagonist that is an antibody specific for L-selectin are administered. Other combinations of L-selectin antagonists will be readily apparent from the instant disclosure.

In some embodiments, an L-selectin antagonist is administered during the entire course of treatment with a second therapeutic agent. In other embodiments, an L-selectin antagonist is administered for a period of time that is overlapping with that of the treatment with the second therapeutic agent. For example, in some embodiments, a subject treatment method involves administering an L-selectin antagonist; and a steroid. Thus, e.g., the L-selectin antagonist treatment can begin before the steroid treatment begins and end before the steroid treatment ends; the L-selectin antagonist treatment can begin after the steroid treatment begins and end after the steroid treatment ends; the L-selectin antagonist treatment can begin after the steroid treatment begins and end before the steroid treatment ends; or the L-selectin antagonist treatment can begin before the steroid treatment begins and end after the steroid treatment ends.

Suitable second therapeutic agents include, but are not limited to, steroids, e.g., hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, conisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, a 21-aminosteroid (a “lazaroid”); a non-steroidal anti-inflammatory drug (NSAID); an antioxidant; a ganglioside; a calcium channel blocker; an inhibitor of lipid peroxidation; a blocker of caspase activation; a glutamate receptor antagonist; an agent that interferes with matrix proteoglycans, e.g., chondroitin sulfate, which agents include, e.g., chondroitinase A and/or B and/or C; an agent that inhibits chondroitin sulfate biosynthesis; inhibitor of myelin-associated glycoprotein (MAG) (see, e.g., U.S. Pat. No. 6,399,577; where suitable MAG inhibitors include a free sialic acid-bearing sugar, a modified derivative of sialic acid attached to a sugar, a sialic acid-bearing sugar attached to a protein or lipid carrier molecule, a modified sialic acid-bearing sugar attached to a protein or lipid carrier molecule, and a sialic acid glycopeptide), Nogo, an OmGp, or their signaling pathways involved in impeding axon growth (see, e.g., (David, S., and Lacroix, S. (2003). Molecular approaches to spinal cord repair. Annu Rev Neurosci 26, 411-440.); neurotrophic factors such as neuroregulin (David, S., and Lacroix, S. (2003). Molecular approaches to spinal cord repair. Annu Rev Neurosci 26, 411-440), neurotrophin-3, neurotrophin-4, brain derived neurotrophic factor, basic fibroblast growth factor, ciliary neurotrophic factor, nerve growth factor, etc.; neural or hematopoietic stem cells; olfactory unsheathing cells; and the like.

A number of 21-aminosteroids have been described in the literature; any known 21-aminosteroid that is effective to treat spinal cord injury is suitable for use. Suitable lazaroids, including, but not limited to, U74389F, U83836E, U74500A, U74006F, U78517F, U78517G, U-78518E, U-78518F, U-78000E, U-75412E, U-75412A, U-74006F, U-74389G, U-74389F, U-77372E, U-74915, U-75014E, and U-75013E, and the like. A variety of 21-aminosteroid compounds have been described in the literature. See, e.g., U.S. Pat. No. 5,614,515; U.S. Pat. No. 6,514,955; WO 87/01706; Durmaz et al. (1999) Pathology & Oncology Research, Vol 5, Nr 3, 223-228; Buttgereit et al. (1995) J. Pharm. Exp. Ther. 275:850; Jacobsen et al. (1990) J. Med. Chem. 33:1145-1151; Hall et al. (1987) J. Neurosurg. 68:456-461; Haynes et al. (1970) Amer. J. Physiol. 259:H144-H148; Zhao et al. (1996) Journal of Neuroscience Research 45: 282-288; Thomas et al. (1993) Biochem. Pharmacol. Vol. 45:241-251.

A lazaroid is generally administered in a single intravenous dose ranging from about 0.1 to about 10.0 mg per kilogram of body weight, or in single oral doses of from about 1 to about 30 mg per kilogram of body weight for every day of therapy. A steroid such as dexamethasone is generally administered in a dosage of from about 10 mg/day to about 100 mg/day.

In some embodiments, methylprednisolone is administered using the following regimen: 5.6 mg/kg for the first 15 minutes; pause 45 minutes; then administer 1 mg/kg/hr thereafter. Methylprednisolone is frequently provided in a solution containing 0.25, 2.5, or 5 mg/mL in 5% dextrose injection or 0.9% sodium chloride. Methylprednisolone sodium succinate (Solu-Medrol) may be administered intravenously at a dosage of 30 mg/kg infused over 10-20 minutes, then intravenously at a dosage of 5.4 mg/kg/hr for 23 hours.

Containers, Devices and Kits

The present invention provides a container comprising an L-selectin antagonist; and devices comprising the container(s). The invention further provides a kit comprising a formulation comprising a unit dosage form of an L-selectin antagonist in a container, and a label that provides instructions for use of the kit.

Suitable containers include those adapted for administration by subcutaneous injection, including a syringe (for use with a needle), an injector pen, and the like. In some embodiments, a subject agonist is administered with a pen injector (e.g., a medication delivery pen), a number of which are known in the art. Exemplary devices which can be adapted for use in the present methods are any of a variety of pen injectors from Becton Dickinson, e.g., BD™ Pen, BD™ Pen II, BD™ Auto-Injector; a pen injector from Innoject, Inc.; any of the medication delivery pen devices discussed in U.S. Pat. Nos. 5,728,074, 6,096,010, 6,146,361, 6,248,095, 6,277,099, and 6,221,053; and the like. The medication delivery pen can be disposable, or reusable and refillable. Also suitable for use is an Intraject® needle-free injection system (Aradigm Corp.).

Suitable containers also include those suitable for use with an implantable device. For example, a container is in some embodiments a reservoir for use with an implantable device. Also suitable for use are containers suitable for use with an injection device, e.g., a needle and syringe, e.g., suitable for intraspinal or intrathecal injection.

In some embodiments, a subject device comprises: i) an infusion pump, which infusion pump includes a container comprising a liquid formulation comprising an L-selectin antagonist; and an intraspinal catheter. In some embodiments, a subject device comprises: i) an infusion pump, which infusion pump includes a container comprising a liquid formulation comprising an L-selectin antagonist; ii) an intraspinal catheter; and iii) an external programmer to control the rate of delivery of the formulation. The pump is operably connected to the intraspinal catheter in such a manner that formulation is pumped from the container through the catheter and to the site of spinal cord injury.

In some embodiments, the invention provides a container that includes a single dosage of an L-selectin antagonist containing an effective amount of the L-selectin antagonist in a dosage form for injecting at or near the site of a spinal cord injury. In some embodiments, the invention provides a pre-filled syringe that includes a single dosage of an L-selectin antagonist containing an effective amount of the L-selectin antagonist in a dosage form for injecting at or near the site of a spinal cord injury. In other embodiments, the invention provides a device suitable for injection at or near the site of a spinal cord injury, the device including a container that includes a single dosage of an L-selectin antagonist containing an effective amount of the L-selectin antagonist.

In other embodiments, the invention provides a device suitable for injection at or near the site of a spinal cord injury, the device including a container that includes a single dosage of an L-selectin antagonist containing an effective amount of the L-selectin antagonist; and a container that includes a single dosage containing an effective amount of a steroid suitable for treating a spinal cord injury. In some embodiments, the device includes a pump for introducing a formulation containing the active agent(s) into a site at or near the site of spinal cord injury.

The present invention provides kits for use in carrying out a subject method. A subject kit generally includes a device for administering an active agent(s) to an individual in need thereof, where the device includes a container comprising a unit dosage form of an L-selectin antagonist. In some embodiments, the device will further include an additional container that comprises a second therapeutic agent, e.g., a steroid (e.g., dexamethasone, methylprednisolone, etc.). In many embodiments, a subject kit will further include instructions for practicing the subject methods or means for obtaining the same (e.g., a website URL directing the user to a webpage which provides the instructions), where these instructions are typically printed on a substrate, which substrate may be one or more of: a package insert, the packaging, reagent containers and the like.

Subjects Suitable for Treatment

Subjects suitable for treatment with a subject method include individuals who have suffered a traumatic spinal cord injury, including e.g., individuals who have suffered a traumatic spinal cord injury as a result of a collision in a moving vehicle; individuals who have suffered a traumatic spinal cord injury during a sporting event or during sport training; individuals who have suffered a traumatic spinal cord injury as a result of a fall; individuals who have suffered a traumatic spinal cord injury during the course of a military engagement; individuals who have suffered a traumatic spinal cord injury as a result of a gunshot wound, a knife wound, or other type of sharp object or blunt object trauma; and the like.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Effect of L-Selectin on Recovery from Traumatic Spinal Cord Injury

Experimental Design

Generation of the Experimental Models

Surgical Procedures

All procedures were performed according to protocols approved by the University of California Committee on Research (San Francisco, Calif.). L-selectin null and wild type littermates were generated bred on a C57BL/6 background. The wild type mice were obtained from the negative littermates of the backcrosses into the C57Bl6 background. These mice have a normal lifespan and there are no overt phenotypic differences between the null and wild type mice. All studies, described below were conducted in a blinded fashion.

Adult, male mice (4-6 months of age), were anesthetized with 2.5% Avertin (0.02 ml/g bw, i.p.) and maintained at 37° C. throughout the experiment by using a warming blanket placed under the animal. A contusive injury was performed based upon modifications of procedures originally described by Kuhn and Wrathal (Kuhn and Wrathall, 1998). Briefly, using aseptic techniques the spinous process and laminae of T8 were removed and a circular region of dura, approximately 2.4 mm in diameter, was exposed. After stabilization of the vertebral column, a 3 gm weight was dropped 5.0 cm onto the exposed dura. After injury, the overlying skin was closed with wound clips. Postoperative care included manual expression of each animal's bladder until recovery of reflex emptying.

Functional Assessments

Locomotor recovery was assessed using an open field testing paradigm, the BBB Locomotor Rating Scale, that is based upon a 21 point scale originally developed in the spinal cord injured rat (Basso et al., 1995). This scale assesses 10 distinct categories that range from limb movement to tail position and involve detailed observations of joint movement, stepping, and coordination. Uninjured animals exhibit a locomotor score of “21” whereas animals that exhibit complete hind limb paralysis are scored as a “0”. Animals that are moderately injured typically show recovery over time and exhibit a locomotor score of between 10 and 11 by about 6 weeks post injury (Basso et al., 1995; Basso et al., 1996). Spinal cord injured animals were tested on days 1 and 3 post injury and weekly thereafter for 6 weeks. Each animal was tested within an enclosed arena of clear acrylic (53 cm×108 cm×5.5 cm) that was supported over a mirror. Positioning of the limbs and locomotion was then observed by either directly or indirectly (via the mirror) viewing the animal.

In addition to locomotor recovery, animals were evaluated with regard to their ability to traverse a grid. This test was chosen because it assesses fine movement of the digits, a function that is controlled in part by the corticospinal tracts. Each animal was placed on a testing arena consisting of a wire grid, 51 cm in length and divided into 1.5×1.5 cm divisions. The ability to grasp the wire grid with each of the hindpaws was determined as the animal traversed the testing arena. The grid score represents the number of times each animal's hindlimbs grasps a wire grid.

Histochemistry

Animals were euthanized at 42 days post-injury and perfused with 50 mL 4% paraformaldehyde (PFA—0.4 g PFA in 50 mL PBS, pH=7.4). The spinal cords were removed, postfixed, and cryoprotected in 20% sucrose for 4 days. Cords were then blocked and frozen at −80° C. until sectioning. 20 μm sections were made on a cryostat, and serial sections, 500 μm apart, were chosen for staining with Luxol Fast Blue (LFB), an indicator of white matter. Residual white matter was selected for analysis. Residual white matter is the best single measurement for characterizing the degree of injury in the contused spinal cord and is predictive of motor recovery (Noble and Wrathall, 1989). Serial sections were selected from the area of maximal damage. That section, representing the most overt loss of white matter, was defined as the lesion epicenter. This section as well as sections 100 and 200 μm rostral and 100 and 200 μm caudal were selected for quantitative analysis of the residual white matter. Each section was examined at the light microscopic level and the area of residual white matter was defined using Neurolucida software (Microbrightfield, Pa.). The percent of residual white matter relative to the cross sectional area was determined for each section. These values were then averaged for each animal.

Statistical Analysis

The mean values for locomotor recovery, performance on a grid, and area of residual white matter were compared between groups using Students T test. Statistical significance was defined at p<0.05.

Results

Motor recovery is significantly improved in the spinal cord injured, L-selectin knockout as compared to the wildtype.

Locomotor performance was evaluated at 1 and 3 days post injury and weekly thereafter for 6 weeks. Recovery was based upon a 21 point scale where 0 represents complete paralysis of the hindlimbs and a score of 21 represents normal locomotion. Importantly, animals that score 10 or higher are able to take weight bearing steps whereas animals scoring less than 10 are unable to step and at best are able to move their hindlimbs in a swimming-like motion referred to as “sweeping”.

There were statistically significant time trends (quadratic, p=0.001) as well as level difference between the knockout and wildtype groups. The trajectories of recovery, however, were similar between the knockout and wildtype animals (p=0.135). Based upon comparisons at each time point (unpaired T Tests), there was a significant improvement in locomotor recovery as early as 3 days post injury (p=0.01) in the knockout as compared to the wildtype animals. The mean score for spinal cord injured knockouts was 2.8 as compared to a mean score of 0.57 for the wildtypes. At this early time point knockout animals showed extensive movement of hip and/or knee joints whereas the wildtypes either showed no observable hindlimb movement or slight movement of the hip and/or knee. Although both groups showed some recovery of locomotor function over time, there was greater restoration of function in the knockout group. By 6 weeks post injury, spinal cord injured knockouts exhibited a significant improvement in motor recovery (mean value of 11.6) as compared to wildtypes (mean value of 8.6) (p=0.001). The knockout animal showed frequent to consistent weight supported steps whereas the wildtype animal was limited to sweeping-like movements of the hindlimb.

The above test of locomotor function does not assess finer movements that involve the digits. These movements are primarily controlled by the corticospinal tracts. To determine if the presence of L-selectin leads to impairment of this type of movement, spinal cord injured animals of both genotypes were evaluated as they traversed a wire grid. This task involves grasping and releasing each bar of the grid as the animal traverses the testing arena. An uninjured animal will make functional contact, as defined by grasping and releasing each bar, with every grid in the arena. Severely spinal cord injured animals are unable to grasp a bar. Rather they drag their hindlimbs across the arena. In contrast, moderately injured animals will grasp some of the bars in the arena. It was found that both wildtype and L-selectin knockout animals were able to take some functional steps in the arena. However, knockout animals were significantly more successful at this task than wildtype animals (p=0.022).

Histologically assessable white matter damage is significantly attenuated in the spinal cord injured L-Selectin knockout as compared to the wildtype.

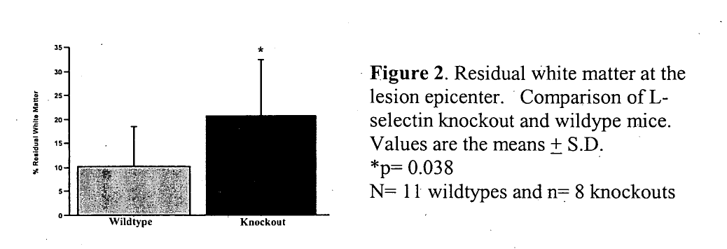

An analysis was conducted to determine whether there is a morphologic correlate to the improved motor recovery in the injured L-selectin knockout animal. The area of residual white matter at the lesioned epicenter was significantly greater in the knockout as compared to the wildtype animals (FIG. 2).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for reducing cell-mediated demyelination of long descending fiber tracts in an individual following mechanical injury to the spinal cord of the individual, the method comprising administering an effective amount of an L-selectin antagonist to the individual.

2. A method for improving locomotor recovery and/or fine motor movement in an individual following spinal cord injury, the method comprising administering an effective amount of an L-selectin antagonist to the individual.

3. The method of claim 1 or claim 2, wherein the L-selectin antagonist is administered to a site at or near the site of spinal cord injury.

4. The method of claim 1 or claim 2, wherein the L-selectin antagonist is administered orally.

5. The method of claim 1 or claim 2, wherein the L-selectin antagonist is administered intravenously.

6. The method of claim 1 or claim 2, wherein the L-selectin antagonist is an antibody specific for L-selectin, wherein the antibody inhibits binding of the L-selectin to an L-selectin ligand in the central nervous system myelin.

7. The method of claim 1 or claim 2, wherein the L-selectin antagonist comprises a peptide that inhibits binding of the L-selectin antagonist to an L-selectin ligand in the central nervous system myelin.

8. The method of claim 1 or claim 2, wherein the L-selectin antagonist is an antibody specific for the central nervous system myelin L-selectin ligand, wherein the antibody inhibits binding of the L-selectin to an L-selectin ligand in the central nervous system myelin.

9. The method of claim 1 or claim 2, wherein the L-selectin antagonist is a soluble form of an L-selectin ligand.

10. The method of claim 9, wherein the L-selectin antagonist is a soluble form of PSGL-1.

11. The method of claim 9, wherein the L-selectin antagonist is a soluble form of endoglycan.

12. The method of claim 9, wherein the L-selectin antagonist is a sulfatide.

13. The method of claim 1 or claim 2, wherein the L-selectin antagonist is a fragment of a central nervous system myelin L-selectin ligand, wherein the fragment inhibits binding of the L-selectin antagonist to the ligand.

14. The method of claim 1 or claim 2, wherein the L-selectin antagonist is an agent that induces shedding of the L-selectin from the surface of a cell that mediates spinal cord demyelination.

15. The method of claim 1 or claim 2, wherein the L-selectin antagonist is a small molecule that directly inhibits binding of an L-selectin to an L-selectin ligand in central nervous system myelin.

16. The method of claim 1 or claim 2, wherein the L-selectin antagonist is administered intraspinally.

17. The method of claim 1 or claim 2, wherein the L-selectin antagonist is administered within 1 hour following traumatic injury to the spinal cord.

18. The method of claim 1 or claim 2, further comprising administering an effective amount of a steroid.

19. The method of claim 18, wherein the steroid is selected from dexamethasone and methylprednisolone.

20. A pharmaceutical composition in a unit dosage form for treating or ameliorating neurological disorders that accompany spinal cord injuries, the composition comprising:
a) a therapeutically effective amount of an L-selectin antagonist;
b) a steroid effective for the amelioration of neurological symptoms associated with spinal cord injuries; and
c) a pharmaceutically acceptable carrier or diluent.

21. The pharmaceutical composition of claim 19, wherein said steroid is methylprednisolone or dexamethasone.

22. A pharmaceutical composition in a unit dosage form for treating or ameliorating neurological disorders that accompany spinal cord injuries, the composition comprising:
a) a therapeutically effective amount of an L-selectin antagonist;
b) an agent that interferes with matrix proteoglycan, wherein the agent effective for the amelioration of neurological symptoms associated with spinal cord injuries; and
c) a pharmaceutically acceptable carrier or diluent.

23. The pharmaceutical composition of claim 21, wherein the agent that interferes with matrix proteoglycan is selected from a chondroitinase, an inhibitor of chondroitin sulfate biosynthesis, and a sulfatase that desulfates chondroitin sulfate.

24. A device suitable for injecting a formulation at or near a site of spinal cord injury, the device comprising:
a) a container comprising a formulation which comprises i) an effective amount of an L-selectin antagonist and ii) a pharmaceutically acceptable excipient; and
b) a needle for injecting the formulation at or near a site of spinal cord injury.

25. The device of claim 24, further comprising a container comprising a formulation, the formulation comprising i) an effective amount of a steroid; and ii) a pharmaceutically acceptable excipient.

26. A device suitable for administering a formulation at or near a site of spinal cord injury, the device comprising:
a) a pump that includes a container comprising a formulation which comprises i) an effective amount of an L-selectin antagonist and ii) a pharmaceutically acceptable excipient; and
b) an intrathecal catheter operably connected to the pump.


SEQUENCE LISTING (1 sequence)

SEQ ID NO: 1 - protein, 372 aa, homo sapien [sequence omitted]